(19) United States
(12) Patent Application Publication
(10) Pub. No.: US 2025/0008991 A1
(43) Pub. Date: Jan. 9, 2025

(54) PROCESS FOR BLEACHING ALGAL BIOMASS BY LIGHT-EXPOSURE

(71) Applicant: NEXTO SOCIETA' A RESPONSABILITA' LIMITATA SEMPLIFICATA, Milano (MI) (IT)

(72) Inventors: Giuliana D'IPPOLITO, Milano (MI) (IT); Maria DI Muro, Milano (MI) (IT)

(21) Appl. No.: 18/705,905

(22) PCT Filed: Oct. 28, 2022
(86) PCT No.: PCT/IB2022/060388

§ 371 Date: Apr. 29, 2024

(30) Foreign Application Priority Data
Oct. 29, 2021 (IT) 102021000027845

(51) Int. Cl. A23L 5/49; A23L 17/00; A23L 17/60

(52) U.S. Cl. CPC A23L 5/49; A23L 17/60; A23L 17/65

(57) ABSTRACT

The process for bleaching algal biomass comprises at least the following phases of:—supply of at least one algal biomass to be bleached;—mixing of the algal biomass with an aqueous solution to obtain a suspension;—light exposure of the suspension to achieve bleaching.

### TECHNICAL FIELD

The present invention relates to a process for bleaching algal biomass.

### BACKGROUND ART

It is well known that there is a particularly strong need in many areas of industrial and commercial activities to renew products and to constantly search for new ones in order to distinguish them more from those of competitors.

This need is particularly felt in the food sector where the search for new nutritional sources is constantly evolving.

In this regard, the 21^{st} century has been characterized by continuous scientific advances in nutrition. Indeed, the discovery of new nutrients together with the writing of guidelines for healthy and proper nutrition and the concept of a balanced diet have accompanied the evolution of food dynamics.

In addition to this, the approval of European regulations on novel foods (2015/2283 of Nov. 25, 2015) and on organic production of food and food ingredients (2016/673 of Apr. 29, 2016), has made possible the use of new ingredients and food products derived from non-traditional sources such as, e.g., algae, i.e., unicellular and multi-cellular, photosynthetic organisms living in aqueous or moist environments, also grown in mixotrophy and heterotrophy. Phytoplankton, microalgae, diatoms and cyanobacteria are also considered included.

Under EU regulations, any food that has not been consumed “to a significant extent” before May 1997 is to be considered a novel food.

This category comprises novel foods, foods from new sources, new substances used in food products as well as new ways and technologies for food production.

In addition to health and nutritional issues (e.g., oils rich in omega-3 fatty acids derived from krill), the main reason for the search for new foods lies in the growing demand for protein sources needed to meet the demands of the human population.

Proteins of animal origin make up about 40% of world consumption. The FAO has predicted a significant increase in their demand by 2050, with enormous repercussions for global environmental and economic sustainability.

Acknowledging this alarm, the European Green Deal, i.e., the set of policy initiatives proposed by the European Commission with the overall goal of achieving climate neutrality in Europe by 2050, has set as one of its priorities the safe production, supply and consumption of alternative and sustainable sources of protein, thus contributing to the transformation of food systems to provide climate co-benefits such as mitigation, adaptation and biodiversity, in favor of healthy nutrition and poverty reduction.

In this context, algae belong to a large and diverse group of aquatic organisms that use photosynthesis like terrestrial plants. Micro-algae are unicellular organisms that can grow in a wide range of environmental conditions.

Thanks to their versatility, they are already widely used for technological and industrial purposes.

Products obtained from the cultivation of micro-algae or photosynthetic organisms are considered an interesting source for the development of new foods.

In addition, such products can be used to increase the nutritional values of conventional foods.

At present, about 30% of the world's algae production is sold for animal feed, but many micro-algae (e.g., Odontella aurita, Chlorella spp., Dunaliella salina, and Aphanizomenon flosaqua, Chlorella vulgaris, Haematococcus pluvialis, Dunaliella salina, Arthrospira platensis) have already been approved and are sold for human food use.

In current food production, micro-algal flours are used as adjuvants in the formulation of high value-added products in m/m percentages ranging from 0.2 to 0.6%.

In particular, micro-algal flours used as ingredients in food processing are rich in nutrients such as: protein, long-chain polyunsaturated fatty acids, carotenoids, vitamins, minerals and polyphenols or other bioactive molecules.

The market based on algae products is expected to reach $44.7 billion by 2023, with an annual growth rate greater than 5% over the period 2016-2023.

However, Europe lacks a tradition and history of micro-algae consumption, contrary to what is common in Southeast Asian countries, complicating therefore consumer acceptability of such products.

In fact, although growing rapidly, the use of algal flours in human nutrition is currently hindered by technological limitations of their processing procedures that do not remove their odor, taste and do not allow their color to be effectively changed, reducing their potential for use as a replacement for traditional industrial protein ingredients such as eggs and milk.

In fact, the typical brackish odor and taste of algae along with its characteristic dark color remain even after the above treatments, thus greatly complicating its spread among consumers.

In this regard, it is well known that the sensory analysis of food products, made from untreated micro-algal flour, such as bread, cookies or pasta preferentially turns to a low concentration of this ingredient in order to limit both the green color, odor and unpleasant taste associated with the fish species that inevitably is preserved and with the high concentration of carotenoids.

### DESCRIPTION OF THE INVENTION

The main aim of the present invention is to devise a process for bleaching algal biomass which allows the color, odor and taste of algal biomass to be changed while keeping its nutritional properties.

Within this aim, one object of the present invention is to devise a process for bleaching algal biomass which allows obtaining white, odorless and tasteless flour.

Another object of the present invention is to devise a process for bleaching algal biomass which allows obtaining low-carotenoid content flour.

A further object of the present invention is to devise a process for bleaching algal biomass which can overcome the aforementioned drawbacks of the prior art within the framework of a simple, rational, easy and efficient to use as well as cost-effective solution.

The aforementioned objects are achieved by this process for bleaching algal biomass having the characteristics of claim 1.

In addition, the aforementioned objects are achieved by flour, obtained by the process in accordance with this invention, having the characteristics of claim 16.

Additionally, the aforementioned objects are achieved by the use of flour, obtained from the process in accordance with this invention, having the characteristics of claim 17.

### Embodiments of the Invention

The process for bleaching algal biomass comprises at least the following phases of:
supply of at least one algal biomass to be bleached;
mixing of algal biomass with an aqueous solution to obtain a suspension; and
light exposure of the suspension to achieve bleaching.

Preferably, the algal biomass to be bleached is obtained from at least one of: algae, micro-algae, cyanobacteria and photosynthetic microorganisms. For example, the aforementioned photosynthetic microorganisms comprise photosynthetic bacteria.

Advantageously, the algal biomass to be bleached is obtained from at least one of: Nannochloropsis salina, Thalassiosira rotula, Thalassiosira pseudonana, Thalassiosira weissflogii, Phaeodactylum tricornutum, Chloroficea tetraflagellata, Nanofrustulum shiloi, Skeletonema marinoi, Dunaliella tertiolecta, Cylindrotheca closterium, Dytilum brightwellii, Botryococcus braunii, Botryococcus breacea, Phaeocystis globosa, Chlorella vulgaris, Cyclotella cryptica, Arthrospira platensis.

It is specified that the term “algal biomass” relates indistinctly to a biomass obtained from a single algal species or, alternatively, from two or more algal species.

Additionally, it is specified that the term “algal biomass” relates indistinctly to algae, micro-algae, cyanobacteria and photosynthetic microorganisms.

In more detail, this expression relates to both fresh algal biomass, i.e., native and wet as it has not undergone treatment aimed at altering its chemical/physical characteristics, and lyophilized algal biomass.

In accordance with a preferred embodiment of the process, the algal biomass to be bleached is in lyophilized form.

The phase of mixing is carried out by introducing the algal biomass to be bleached within a container containing the aqueous solution.

Such an aqueous solution comprises water. Preferably, such aqueous solution consists of water.

Advantageously, the aforementioned aqueous solution consists of distilled water.

Additionally, the aforementioned container comprises a filtering cover element adapted to preserve the suspension from environmental contamination and/or introduction of particulate matter.

The cover element is of the type, e.g., of a cap provided with a gas exchange filter.

Preferably, the suspension has a concentration by weight of the algal biomass to be bleached, evaluated with respect to the total weight of the suspension, of less than 5%.

In detail, the algal biomass to be bleached is solubilized in distilled water with a suspension volume/container surface area ratio of 1 ml/cm² and a suspension volume/container volume ratio of 0.2 ml/cm³.

In addition, the suspension has a salt concentration equal to 0 g/L.

In this regard, it should be pointed out that when the algal biomass is fresh, prior to the phase of supply, the process in accordance with the present invention comprises at least one phase of desalting the algal biomass to be bleached.

The phase of desalting is carried out by means of a washing step of the aforementioned algal biomass to be bleached.

In more detail, this washing step is repeated until desalinated algal biomass is obtained.

Advantageously, the washing step of the algal biomass to be bleached is repeated three times.

Following this, the process comprises at least one phase of stirring the suspension.

The stirring phase is carried out by means of a rotating stirrer having a rotational speed of more than 30 rpm.

Preferably, the rotational speed is substantially equal to 40 rpm.

The aforementioned rotational speed is necessary so that algal biomass will not be deposited at the bottom of the container.

In more detail, the type of stirrer and the speed thereof are key aspects that contribute to the bleaching efficiency of the algal biomass. It is, in fact, of paramount importance that stirring takes place in such a way as to move the algal biomass and allow it to be evenly exposed to light radiation.

By way of example, the stirrer may be of the mechanical type, i.e., a motor-driven paddle stirrer of a type known to the technician in the field.

It cannot, however, be ruled out from the scope of this disclosure that, in accordance with an alternative embodiment of the process according to the invention, the stirring phase is carried out by air.

Preferably, the air stirring phase is carried out by delivering compressed air inside the suspension.

According to the invention, the phase of light exposure is carried out at an emission wavelength of less than 900 nm.

Preferably, the aforementioned emission wavelength is of between 300 nm and 800 nm.

The phase of light exposure is carried out by setting up lamps outside the container or, alternatively, by exposure to sunlight, the emission wavelength ranging between 100 nm and 400 nm.

In this regard, it should be noted that the container is made at least partly of transparent material.

Such lamps have a luminous flux of 2800 lm.

Preferably, such lamps have a color temperature substantially equal to 4000K.

The aforementioned lamps are associated with a supporting frame that is external to the container and adapted to allow the homogeneous irradiation of the suspension contained therein.

In addition, the process comprises at least one phase of heating the suspension.

Advantageously, the heating phase is carried out at a temperature above 30° C.

Preferably, the temperature is of between 50° C. and 60° C.

As an example, the container has an outer jacket, connected to an inlet and outlet water system, which allows the suspension to be thermo-regulated.

Preferably, the heating phase is carried out simultaneously with the phase of light exposure.

It cannot, however, be ruled out from the scope of this disclosure that the heating phase is carried out prior to the phase of light exposure.

Advantageously, the heating phase lasts substantially 4 hours.

The interruption of the bleaching phase is carried out at the colorimetric change of the suspension from the original color to white-beige.

This means that the moment the suspension is free of colored pigments, the bleaching phase is interrupted.

The expression “free of colored pigments” relates to the fact that the suspension is achromatic or with a color very close to white.

Next, the process comprises a recovery phase of the bleached suspension.

This recovery phase is carried out through an outlet duct associated with the container.

Finally, the process comprises at least one freeze-drying phase of the bleached suspension.

It cannot be ruled out from the scope of this disclosure that prior to the freeze-drying phase, the bleached suspension is subjected to freezing at a temperature below −20° C.

In a second aspect, the present invention relates to algal flour obtained by the aforementioned process and containing a protein fraction present in a concentration by weight, evaluated with respect to the total weight of the flour, greater than 5%, preferably greater than 10%.

Finally, in a third aspect, the present invention relates to the use of algal flour obtained from the process in accordance with the present invention in food products.

It has in practice been ascertained that the described invention achieves the intended objects.

It is emphasized that the special expedient of providing the synergistic combination of a phase of light exposure and a phase of heating makes it possible to achieve bleaching of the algal biomass while maintaining its nutritional properties.

More in detail, the technical expedient of providing the suspension of algal biomass, either native or lyophilized, to be bleached in an aqueous suspension allows its nutritional properties to remain unaltered, while avoiding the loss of protein components as a result of centrifugation treatments.

1. Process for bleaching algal biomass, characterized by the fact that it comprises at least the following phases of:
supply of at least one algal biomass to be bleached;
mixing of said algal biomass with an aqueous solution to obtain a suspension;
light exposure of said suspension to achieve bleaching.

2. Process according to claim 1, characterized by the fact that said phase of light exposure is carried out at an emission wavelength of less than 900 nm.

3. Process according to claim 1, characterized by the fact that said phase of light exposure is carried out at an emission wavelength of between 300 nm and 800 nm.

4. Process according to claim 1, characterized by the fact that it comprises at least one phase of heating of said suspension.

5. Process according to claim 1, characterized by the fact that said phase of heating is carried out at a temperature above 30° C.

6. Process according to claim 1, characterized by the fact that said algal biomass to be bleached is obtained from at least one of:
algae, micro-algae, cyanobacteria and photosynthetic microorganisms.

7. Process according to claim 1, characterized by the fact that said algal biomass is obtained from at least one of: Nannochloropsis salina, Thalassiosira rotula, Thalassiosira pseudonana, Thalassiosira weissflogii, Phaeodactylum tricornutum, Chloroficea tetraflagellata, Nanofrustulum shiloi, Skeletonema marinoi, Dunaliella tertiolecta, Cylindrotheca closterium, Dytilum brightwellii, Botryococcus braunii, Botryococcus breacea, Phaeocystis globosa, Chlorella vulgaris, Cyclotella cryptica, Arthrospira platensis.

8. Process according to claim 1, characterized by the fact that said algal biomass to be bleached is in lyophilized or native form.

9. Process according to claim 1, characterized by the fact that it comprises at least one phase of stirring of said suspension.

10. Process according to claim 1, characterized by the fact that said stirring phase is carried out by means of a rotating mechanical stirrer having a rotational speed of more than 30 rpm.

11. Process according to claim 1, characterized by the fact that said stirring phase is carried out by air.

12. Process according to claim 1, characterized by the fact that said suspension has a low salt concentration or is completely free of salt equal to 0 g/L.

13. Process according to claim 1, characterized by the fact that it comprises at least one desalting phase of said algal biomass to be bleached, said desalting phase being prior to said supply phase.

14. Process according to claim 1, characterized by the fact that said suspension has a concentration by weight of said algal biomass to be bleached, evaluated with respect to the total weight of the suspension, of less than 5%.

15. Process according to claim 1, characterized by the fact that it comprises at least one freeze-drying phase of said bleached suspension.

16. Algal flour obtained by the process according to claim 1, having a protein fraction present in a concentration by weight, evaluated with respect to the total weight of the flour, of more than 10%, preferably more than 5%.

17. Use of the algal flour according to claim 15 in food products.
